(19) United States
(12) Patent Application Publication
(10) Pub. No.: US 2026/0102083 A1
(43) Pub. Date: Apr. 16, 2026

(54) MEDICAL SYSTEM AND METHOD FOR MONITORING THE STORAGE CONDITIONS THEREOF

(71) Applicants: Roche Diagnostics GmbH, Mannheim (DE); Roche Diabetes Care GmbH, Mannheim (DE)

(72) Inventors: Alexander Steck, Hirschberg (DE); Kirill Sliozberg, Mannheim (DE)

(21) Appl. No.: 19/422,786

(22) Filed: Dec. 17, 2025

Related U.S. Application Data
(63) Continuation of application No. PCT/EP2024/066929, filed on Jun. 18, 2024.

(30) Foreign Application Priority Data
Jun. 21, 2023 (EP) 23180645.6
Jun. 26, 2023 (EP) 23181445.0

(51) Int. Cl. A61B 5/145; A61B 5/00

(52) U.S. Cl. CPC A61B 5/14546; A61B 5/6847

(57) ABSTRACT

A medical system and a method for monitoring the storage conditions of at least one medical system are disclosed. The medical system includes at least one analyte sensor for detecting at least one analyte in a bodily fluid, the analyte sensor being configured for at least partial transcutaneous insertion into a body tissue of a user; and at least one disabling device operably connected to the analyte sensor. The disabling device includes at least one monitoring element wherein the monitoring element is configured for undergoing a change in at least one mechanical property when storage conditions of the analyte sensor are outside a specification range. The mechanical property change is configured such that the transcutaneous insertion of the analyte sensor into the body tissue is irreversibly prevented.

### CROSS REFERENCE TO RELATED APPLICATIONS

This is a continuation of application no. PCT/EP2024/066929 filed Jun. 18, 2024, which claims priority to EP 23 180 645.6 filed Jun. 21, 2023, and EP 23 181 445.0 filed Jun. 26, 2023, the disclosures of each of which are hereby incorporated herein by reference.

### BACKGROUND

The present disclosure relates to a medical system and a method for monitoring the storage conditions of the medical system. The medical system may specifically be configured for detecting at least one analyte in a bodily fluid of a user. The medical system and the method, as an example, may be used in the field of continuous monitoring of the analyte in the bodily fluid of the user, more specifically in the field of home care and/or in the field of professional care, such as in hospitals or the like. Other applications, however, are also feasible.

Monitoring certain body functions, more particularly monitoring one or more analyte concentrations such as one or more metabolite concentrations in a bodily fluid of a user plays an important role in the prevention and treatment of various diseases. Such analytes can include by way of example, but not exclusively, glucose, lactate, cholesterol or other types of analytes and metabolites. Without restricting further possible applications, the present disclosure will be described in the following text with reference to glucose monitoring. However, additionally or alternatively, the invention can also be applied to other types of analytes.

In the field of glucose monitoring, medical devices are known comprising chemical and/or biochemical compounds which are sensitive to storage conditions, such as temperature and/or humidity. Thus, monitoring ambient condition of medical devices is generally known.

US 2020/0321094 A1 discloses methods, systems, and devices for administering a medicament to a patient. In one aspect, a system includes an injection pen device in wireless communication with a mobile communication device. The injection pen device includes a housing including a chamber to encase a cartridge containing medicine, a dose setting and dispensing mechanism to set the mechanism to dispense a particular dose of the medicine from the loaded cartridge, a sensor unit to detect a dispensed dose based on positions and/or movements of the dose setting and dispensing mechanism, and an electronics unit in communication with the sensor unit to process the detected dispensed dose and time data associated with a dispensing event and to wirelessly transmit the dose data to a user's device. The mobile communication device provides a software application to provide the user with health information using the processed dose data.

US 2017/0216519 A1 describes a system for dispensing a fluid. The system has a housing having a fixation means to a user and an orientation element; the cartridge formed so as to be held in a given orientation by the orientation element with respect to the housing; and control means to activate the cartridge to eject a fluid. In one aspect, the system is an emergency rescue fluid(s) transdermal delivery system which includes a removable, single use emergency rescue fluid(s) dispensing cartridge, a wearable device into which the emergency rescue fluid(s) dispensing cartridge uniquely, matingly and removably inserts. The system includes a communication pathway between the wearable device and at least one other cloud network node, and/or at least one communication pathway between the wearable device and at least one GPS network node. In another aspect, the system dispenses a perfume or other fragrance.

WO 2007/092637 A2 discloses a patch-sized fluid delivery device including a reusable portion and a disposable portion. The disposable portion may include components that come into contact with the fluid, while the reusable portion may include only components that do not come into contact with the fluid. Redundant systems, such as redundant controllers, power sources, motor actuators, and alarms, may be provided. Alternatively or additionally, certain components can be multi-functional, such a microphones and loudspeakers that may be used for both acoustic volume sensing and for other functions and a coil that may be used as both an inductive coupler for a battery recharger and an antenna for a wireless transceiver. Various types of network interfaces may be provided to allow for remote control and monitoring of the device.

U.S. Pat. No. 8,905,965 B2 describes a medical remote controller device. The device includes a display and at least one input switch dedicated to bolus delivery, wherein a bolus delivery is programmed when the input switch receives an input and wherein the number of inputs received by the input switch determines the amount of bolus to be delivered.

US 2018/0204636 A1 discloses a computer-implemented method including establishing a communications link, via a short-range wireless protocol, between a mobile computing device and a medicament delivery device. A user input selecting a motion profile of the medicament delivery device is then received in response to an input prompt. A wireless signal is received from the medicament delivery device, the wireless signal associated with an actual motion profile of the medicament delivery device. A notification is produced to indicate a motion difference between the actual motion profile and the target motion profile. In some embodiments, the method optionally includes modifying the target motion profile based on the motion profile over a time period of at least one week, the notification indicating a motion difference between the motion profile and the modified target motion profile.

WO 2018/077993 A1 describes systems, methods, and apparatus for a medication delivery device. The device includes a dose selector for selecting an amount of medication to deliver; a first capacitive sensor adjacent the dose selector and operative to detect linear displacement of the dose selector during medication delivery; a screw coupled to the dose selector; a second capacitive sensor adjacent the screw and operative to detect linear displacement of the screw during medication delivery; and a processor coupled to the first and second capacitive sensors and operative to determine an amount of medication actually delivered by the medication delivery device.

In non-medical fields of application, monitoring of ambient condition is known, too. For example, U.S. Pat. No. 7,549,375 B2 describes devices for mitigating the explosive reaction of a munition when it is subject to an external thermal hazard threat. The devices are based on the use of shape memory alloys. In one arrangement, there is device which consists of a connector that is at least in part formed from a shape memory alloy, which typically undergoes large dimensional changes when heated or cooled through a particular transition temperature range. The connector is designed to form a locking engagement, between two components of a munitions casing at one temperature, but when subjected to external heating through the transition temperature range will deform to allow the connector to disengage and thus release the two joined components, allowing any build up of pressure to be released quickly. Advantageously if the co-operative parts of the connector and components are threaded portions, then the locking engagement will be capable of being dismantled during normal servicing of the munition. The co-operative parts of the connector may be integral with the components to be connected. In another arrangement, the device is an annulus and is located around a munitions casing such that upon heating through its transition temperature range will cause the annulus to contract, thereby rupturing the munitions casing, allowing any build up of pressure to be released quickly.

U.S. Pat. No. 10,260,956 B2 discloses an apparatus, system and method for a time temperature indicator (TTI) which is capable of providing a summary of the time and temperature history of a good to which it is coupled, optionally including with regard to providing an indication as to whether one or more temperature thresholds have been breached. According to other embodiments, the TTI specifically provides an indication as to whether a temperature threshold at or around the freeze point has been breached, optionally even without providing a time and temperature history.

Despite the advantages achieved by known methods and devices, several technical challenges remain. Specifically, as outlined above, medical devices, e.g. medical device for glucose monitoring, may comprise chemical and/or biochemical compounds which are sensitive to storage conditions, such as temperature and/or humidity. Appropriate storage conditions are usually provided with the medical device, e.g. indicated thereon. In principle, storage conditions of those medical devices at a reseller's site may be considered proper, whereas end user may be not aware of the proper storage conditions and/or of the importance of ensuring proper storage conditions. Such medical devices, e.g. continuous glucose monitoring (CGM) devices, may lose their sensitivity towards the analyte to be detected, e.g. towards glucose, and, thus, may exhibit negative signal drifts due to improper storage conditions, such as storage at elevated temperatures and/or humidity. Further, for factory-calibrated medical devices, sensitivity degradation may lead to a permanent underestimation of analyte concentration, e.g. an underestimation of glucose levels. Thus, there is a need for preventing usage of medical devices which were stored under improper storage conditions.

### SUMMARY

It is desirable to provide devices and methods which at least partially address above-mentioned technical challenges. Specifically, a medical system and a method for monitoring the storage conditions of the medical system shall be proposed which completely prevents usage when stored under improper storage conditions and, preferably, prior to analyte sensor insertion.

This problem is addressed by a medical system and a method for monitoring the storage conditions of at least one medical system, with at least some of the features described herein. Advantageous embodiments which might be realized in an isolated fashion or in any arbitrary combinations are also described throughout the specification.

As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements.

Further, it shall be noted that the terms “at least one”, “one or more” or similar expressions indicating that a feature or element may be present once or more than once typically will be used only once when introducing the respective feature or element. In the following, in most cases, when referring to the respective feature or element, the expressions “at least one” or “one or more” will not be repeated, non-withstanding the fact that the respective feature or element may be present once or more than once.

Further, as used in the following, the terms “preferably”, “more preferably”, “particularly”, “more particularly”, “specifically”, “more specifically” or similar terms are used in conjunction with optional features, without restricting alternative possibilities. Thus, features introduced by these terms are optional features and are not intended to restrict the scope of the claims in any way. The invention may, as the skilled person will recognize, be performed by using alternative features. Similarly, features introduced by “in an embodiment of the invention” or similar expressions are intended to be optional features, without any restriction regarding alternative embodiments of the invention, without any restrictions regarding the scope of the invention and without any restriction regarding the possibility of combining the features introduced in such way with other optional or non-optional features of the invention.

In a first aspect of the present disclosure, a medical system is disclosed. The medical system comprises:
at least one analyte sensor for detecting at least one analyte in a bodily fluid, the analyte sensor being configured for at least partial transcutaneous insertion into a body tissue of a user; and
at least one disabling device operably connected to the analyte sensor, the disabling device comprising at least one monitoring element, the monitoring element being configured for undergoing a change in at least one mechanical property when storage conditions of the analyte sensor are outside a specification range, the mechanical property change being configured such that the transcutaneous insertion of the analyte sensor into the body tissue is irreversibly prevented.

The term “system” as used herein is a broad term and is to be given its ordinary and customary meaning to a person of ordinary skill in the art and is not to be limited to a special or customized meaning. The term specifically may refer, without limitation, to an arbitrary set of interacting or interdependent components parts forming a whole. The system may comprise multiple components, for example at least two components or even more. The at least two components may be handled independently or may be coupled or connectable. The components of the system may interact with each other in order to fulfill at least one common function. Consequently, the term “medical system” as used herein generally may refer, without limitation, to a system as defined above, which is configured for serving at least one medical purpose, i.e. at least one purpose selected from the group consisting of a diagnostic purpose and a therapeutic purpose.

As outlined above, the medical system comprises at least one analyte sensor for detecting at least one analyte in a bodily fluid. The term “analyte” as used herein is a broad term and is to be given its ordinary and customary meaning to a person of ordinary skill in the art and is not to be limited to a special or customized meaning. The term specifically may refer, without limitation, to a chemical and/or biological substance of interest, more specifically to at least one chemical and/or biological substance, taking part in the metabolism of the body of a subject. Exemplarily, the analyte may be a metabolite or a combination of two or more metabolites. As an example, the analyte may be selected from the group consisting of: glucose, lactate, triglycerides, cholesterol. A preferred analyte is glucose. Still, other analytes or combinations of two or more analytes may be detected.

The analyte sensor specifically may be configured for being used in qualitatively and/or quantitatively detecting the at least one analyte. The term “analyte sensor” as used herein is a broad term and is to be given its ordinary and customary meaning to a person of ordinary skill in the art and is not to be limited to a special or customized meaning. The term specifically may refer, without limitation, to a sensor which is capable of qualitatively or quantitatively detecting the presence and/or the concentration of the at least one analyte.

The analyte sensor may be or may comprise at least one electrochemical analyte sensor. The analyte sensor may comprise at least two electrodes. Specifically, the analyte sensor may comprise at least one two-electrode sensor. The two-electrode sensor may comprise precisely two electrodes, such as a working electrode and at least one further electrode such as a counter electrode, e.g. a working electrode and a combined counter/reference electrode. The working electrode may comprise a working electrode pad and, optionally, at least one test chemical disposed thereon. The counter electrode may comprise a counter electrode pad. Additionally and optionally, one or more redox materials may be disposed thereon. The analyte sensor may further comprise one or more leads for electrically contacting the electrodes. The leads may, during insertion or at a later point in time, be connected to one or more electronic components. For example, the leads may already be connected to the electronic components before insertion of the analyte sensor. For example, the analyte sensor may be a needle-shaped or a strip-shaped analyte sensor having a flexible substrate and the electrodes disposed thereon. As an example, the analyte sensor may have a total length of 5 mm to 50 mm, specifically a total length of 7 mm to 30 mm. The term “total length” within the context of the present disclosure relates to the overall length of the analyte sensor which means a portion of the analyte sensor which is inserted and the portion of the analyte sensor which may stay outside of the body tissue. The portion of the analyte sensor which is inserted is also called the in-vivo portion, the portion of the analyte sensor which may stay outside of the body tissue is also called the ex-vivo portion. For example, the in-vivo portion has a length in the range from 3 mm to 12 mm. The analyte sensor may further comprise a biocompatible cover, such as a biocompatible membrane which fully or partially covers the analyte sensor and which prevents the test chemical from migrating into the body tissue and which allows for a diffusion of the bodily fluid and/or the analyte to the electrodes. Other embodiments of electrochemical analyte sensors, such as three-electrode sensors, may be feasible. For example, the three-electrode sensor may comprise, in addition to the working electrode and the counter electrode, a reference electrode. Such analyte sensors are generally known in the art and include continuous glucose sensor systems such as Dexcom's G6 or G7 glucose sensor system, Medtronic's Enlite Glucose sensor or Abbott's Freestyle Libre 2 or 3.

Additionally or alternatively, the analyte sensor may be or may comprise at least one optical analyte sensor. For example, the analyte sensor may comprise a flexible light guide with glucose sensitive coating at its end and/or a tube-like carrier with functional elements at inner or outer walls. Other embodiments of the analyte sensor may be possible too.

The term “bodily fluid” as used herein is a broad term and is to be given its ordinary and customary meaning to a person of ordinary skill in the art and is not to be limited to a special or customized meaning. The term specifically relates to an arbitrary fluid which typically is present in a body or body tissue of a user or a patient and/or which may be produced by the body of the user or the patient. As an example for body tissue, interstitial tissue may be named. Thus, as an example, the bodily fluid may comprise at least one bodily fluid selected from the group consisting of blood and interstitial fluid. However, additionally or alternatively, one or more other types of bodily fluids may be used, such as saliva, tear fluid, urine or other body fluids. During detection of at least one analyte, the bodily fluid may be present within the body or body tissue. Thus, specifically, the analyte sensor may be an in-vivo analyte sensor.

As also outlined above, the analyte sensor is configured for at least partial transcutaneous insertion into a body tissue of a user.

As used herein, the term “transcutaneous insertion” as used herein is a broad term and is to be given its ordinary and customary meaning to a person of ordinary skill in the art and is not to be limited to a special or customized meaning. The term specifically may refer to a process of inserting an object through the body surface or skin of a user, into a body tissue of the user. Thereafter, the object may fully or partially be located in the body tissue and/or under the skin of the user. At least one part of the object, however, may also protrude from the body tissue to the environment. The part intended for insertion into the body tissue may also be referred to as the insertable portion. The term “insertable portion”, thus, may generally refer to a part or component of an element configured to be insertable into an arbitrary body tissue. Other parts or components of the analyte sensor may remain outside of the body tissue, e.g. counter electrode and/or reference electrode or combined counter/reference electrode may remain outside of the body tissue. Preferably, the insertable portion may fully or partially comprise a biocompatible surface, which may have as little detrimental effects on the user or the body tissue as possible, at least during typical durations of use. For this purpose, the insertable portion may be fully or partially covered with at least one biocompatibility membrane layer, such as at least one polymer membrane, for example a gel membrane. The analyte sensor, thus, specifically may be an in-vivo analyte sensor.

The term “in-vivo sensor” as used herein is a broad term and is to be given its ordinary and customary meaning to a person of ordinary skill in the art and is not to be limited to a special or customized meaning. The term specifically may refer, without limitation, to a sensor which is configured for being at least partially implanted into a body tissue of a user.

As further outlined above, the medical system further comprises at least one disabling device operably connected to the analyte sensor. The disabling device comprises at least one monitoring element, the monitoring element being configured for undergoing a change in at least one mechanical property when storage conditions of the analyte sensor are outside a specification range, the mechanical property change being configured such that the transcutaneous insertion of the analyte sensor into the body tissue is irreversibly prevented.

The term “disabling device” as used herein is a broad term and is to be given its ordinary and customary meaning to a person of ordinary skill in the art and is not to be limited to a special or customized meaning. The term specifically may refer to a device configured for fully or partially preventing at least one function of another device or a system. The disabling device, as an example, may disable the at least one function by at least one of a blocking of the function, a destroying of at least one component necessary for performing the function, disengaging at least two components, the engagement of which is necessary for performing the function. Other ways of disabling are also generally feasible.

As further used herein, the term “operably connected” is a broad term and is to be given its ordinary and customary meaning to a person of ordinary skill in the art and is not to be limited to a special or customized meaning. The term specifically may refer to a relation between at least two elements enabling an interaction between the at least two elements, specifically an interaction selected from the group consisting of a mechanical interaction, a thermal interaction, an electrical interaction and an optical interaction.

As further used herein, the term “monitoring element” is a broad term and is to be given its ordinary and customary meaning to a person of ordinary skill in the art and is not to be limited to a special or customized meaning. The term specifically may refer to an element or a combination of elements configured for monitoring, at least one property of a surrounding, and/or of at least one object to be monitored, such as, in the present case, the analyte sensor. The monitoring may relate to at least one property selected from the group consisting of a physical property, a chemical property, a biological property. More specifically, the property may comprise at least one property selected from the group consisting of a temperature and a humidity.

As outlined above, the monitoring element is configured for undergoing at least one change in at least one mechanical property when storage conditions of the analyte sensor are outside a specification range. As used therein, the term “mechanical property” is a broad term and is to be given its ordinary and customary meaning to a person of ordinary skill in the art and is not to be limited to a special or customized meaning. The term specifically may refer to at least one property selected from the group consisting of: a tensile strength; a yield strength, an elastic modulus; a water uptake. As further used therein, the term “storage conditions” is a broad term and is to be given its ordinary and customary meaning to a person of ordinary skill in the art and is not to be limited to a special or customized meaning. The term specifically may refer to at least one item of information characterizing the environmental conditions which at least one object of interest currently is exposed to and/or has been exposed to over at least one predetermined period of time, such as from a point in time in the past until the present point in time, such as from the point in time of manufacturing the object of interest until the present point in time. The environmental conditions, specifically may relate to at least one of a temperature, an absolute humidity and a relative humidity.

As further used herein, the term “specification range” is a broad term and is to be given its ordinary and customary meaning to a person of ordinary skill in the art and is not to be limited to a special or customized meaning. The term specifically may refer to a predetermined or determinable range of at least one parameter or item of information, specifically a range defined by a list of numerical values and/or by at least one numerical interval, e.g. a closed interval, and open interval, or a semi-open interval. The specification range, specifically may define at least one admissible range of values, wherein, if the at least one parameter or item of information is found to correspond to at least one of the values within the admissible range of values, the parameter or item of information is found to be within specification and, otherwise, the parameter or item of information is found to be out of specification. The specification range, as an example, may be predetermined by experiments and/or by analytical considerations. Thus, as an example, the specification range for the storage conditions of the analyte sensor may be determined by one or more parameters, such as at least one of a maximum humidity to which the analyte sensor has been exposed, an average humidity to which the analyte sensor has been exposed, a cumulated humidity over time to which the analyte sensor has been exposed, a maximum temperature to which the analyte sensor has been exposed, an average temperature to which the analyte sensor has been exposed, a cumulated temperature over time to which the analyte sensor has been exposed, such as heat equivalent units or the like. Other ways of determining the specification range are also generally feasible.

As further outlined above, the monitoring element is configured for undergoing a change in at least one mechanical property when the storage conditions of the analyte sensor are outside the specification range. As will be outlined in further detail below, by corresponding examples, the monitoring element specifically may be sensitive to the storage conditions, by, as an example, inherent properties affected by the storage conditions. For this purpose, the monitoring elements may comprise at least one material which undergoes the at least one change when the storage conditions are outside the specification range. The medical system may be configured such that the monitoring element of the disabling device, when the medical system is stored, is exposed to identical or similar storage conditions as the analyte sensor itself, so the state of the monitoring element may be an indicator of the state of the analyte sensor.

As also outlined above, the change in the at least one mechanical property of the monitoring element which occurs when the storage conditions of the analyte sensor are outside the specification range is such that the insertion of the analyte sensor into the body tissue is irreversibly prevented. Various examples of preventing the insertion of the analyte sensor into the body tissue will be given below. The term “irreversibly” as used herein specifically may refer to the fact that the prevention of the insertion may not be undone, as an example, by bringing the storage conditions of the analyte sensor back into a range which is considered to correspond to the specifications, such as by lowering the current temperature to which the analyte sensor is exposed and/or by lowering the humidity to which the analyte sensor is exposed.

The mechanical change of the monitoring element, which occurs when the storage conditions of the analyte sensor are outside a specification range, specifically may be selected from the group consisting of: a change in a tensile strength of the monitoring element; a change in a shape of the monitoring element; an elongation of the monitoring element under load, specifically an elongation of the monitoring element under spring load; a breaking of the monitoring element.

As further outlined above, the specification range may be defined by one or more parameters. Examples are given above. More specifically, the specification range of the storage conditions may be defined by at least one of a predetermined temperature range and a predetermined humidity range. For example, the specification range of the storage conditions may be defined by one of a predetermined temperature range and a predetermined humidity range or by a combination of a predetermined temperature range and a predetermined humidity range.

The disabling device may comprise a plurality of monitoring elements. Even more than one disabling device each having at least one monitoring element may be feasible. The monitoring elements may be embodied identical or different with respect to each other. For example, the disabling device may comprise at least two monitoring elements, wherein at least one of the monitoring elements may be configured for undergoing the change in the mechanical property when storage temperature of the analyte sensor is outside a specification range, wherein at least one other monitoring elements may be configured for undergoing the change in the mechanical property when storage humidity of the analyte sensor is outside a specification range. Other combinations or options are also feasible.

As outlined above, various ways of preventing the insertion of the analyte sensor into the body tissue are generally feasible. Reference may be made to the above-mentioned options of the disabling device. As a specific example, exemplary embodiments of which will be given in further detail below, the disabling device may further comprise at least one counterbalancing element, the counterbalancing element and the monitoring element exerting opposing forces onto at least one part of the disabling device. Therein, the part of the disabling device onto which the opposing forces are exerted may be distinct from the counterbalancing element and the monitoring element or, alternatively, may also fully or partially be identical with one or both of these elements. Further, as used herein, the term “opposing forces” may generally relate to the fact that the monitoring element exerts at least one first force onto the part of the disabling device and that the counterbalancing element exerts at least one second force onto the part of the disabling device, wherein the first force and the second force have force components opposing each other, by pointing into opposing directions. Thus, the first force and the second force may point into opposing directions or may have force components pointing into opposing directions.

The opposing forces which are exerted by the counterbalancing element and the monitoring element may keep the at least one part of the disabling device onto which the opposing forces are exerted in a predetermined place as long as the storage conditions are within the specification range. As soon as the storage conditions are found to be outside the specification range, such as in case at least one of a maximum temperature exposure, an average temperature exposure, a cumulated temperature exposure, a maximum humidity exposure, an average humidity exposure and a cumulated humidity exposure of the analyte sensor exceed one or more threshold values, a counterbalancing of the opposing forces may be disturbed by the change in the at least one mechanical property of the monitoring element, thereby exerting a net force onto the counterbalancing element. By the net force, as an example, the at least one part of the disabling device may be moved in order to prevent the insertion of the analyte sensor. Thus, as an example, a blocking effect may occur by the movement of the at least one part. Additionally or alternatively, the at least one part may also fully or partially be identical with the monitoring element, and the counterbalancing element, when the at least one mechanical property of the monitoring element changes due to the storage conditions of the analyte sensor being outside a specification range, may change the shape of the monitoring element, e.g. by tearing apart the monitoring element when the tensile strength of the monitoring element falls below a threshold. Other exemplary embodiments are feasible.

The counterbalancing element, as an example, may comprise at least one spring element. As an example, at least one metallic and/or at least one plastic spring element may be present in the counterbalancing element. As an example, the monitoring element may be compressed directly or indirectly by the spring element or may be stretched directly or indirectly by the spring element. Alternatively or additionally, the spring element may be compressed directly or indirectly by the monitoring element.

The opposing forces exerted by the counterbalancing element and the monitoring element may be balanced when the analyte sensor has been stored under storage conditions within the specification range. Thus, the balancing of the opposing forces may be an indicator of the fact that the analyte sensor has been stored under storage conditions within the specification range. On the other hand, the opposing forces exerted by the counterbalancing element and the monitoring element may be or may become unbalanced when the analyte sensor has been stored, at least over a period of time, under storage conditions outside the specification range. Thus, the change in at least one mechanical property of the monitoring element due to the fact that storage conditions of the analyte sensor are outside a specification range may lead to an unbalancing of the opposing forces, such as by a loss in tensile strength of the monitoring element, by a change in shape of the monitoring element or by a breaking of the monitoring element. The unbalancing may directly or indirectly prevent the transcutaneous insertion of the analyte sensor, such as by destroying an insertion mechanism and/or by blocking an insertion mechanism. Examples will be given in further detail below.

The at least one part of the disabling device onto which the opposing forces of the counterbalancing element and the monitoring element are exerted may, as an example, be chosen in accordance with the design of the analyte sensor itself and/or an insertion mechanism for inserting the analyte sensor. Thus, as an example, the at least one part of the disabling device onto which the opposing forces of the counterbalancing element and the monitoring element are exerted may comprise at least one mechanical lock configured for moving from an unlocked position into a locked position when the opposing forces of the counterbalancing element and the monitoring element become unbalanced due to a storage of the analyte sensor outside the specification range.

The medical system may further comprise at least one insertion device configured for engaging with the analyte sensor, and for transcutaneously inserting at least a part of the analyte sensor into the body tissue.

As used herein, the term “insertion device” is a broad term and is to be given its ordinary and customary meaning to a person of ordinary skill in the art and is not to be limited to a special or customized meaning. The term specifically may refer to an arbitrary device which is configured for driving a motion of the analyte sensor, through the skin of the user, into the body tissue. For this purpose, the insertion device specifically may comprise at least one actuator configured for directly or indirectly engaging with the analyte sensor and for directly or indirectly exerting a force onto the analyte sensor in order to drive the analyte sensor, through the skin of the user, into the body tissue. The insertion device, as will be outlined in further detail below, may also comprise at least one penetration element, such as at least one cannula and/or at least one needle.

In case at least one insertion device is provided, the disabling device may prevent the transcutaneous insertion of the analyte sensor into the body tissue by the insertion device in various ways, which may be used in isolation or which may also be combined. Thus, in one example, the disabling device specifically may be configured for preventing an engagement of the analyte sensor by the insertion device when the analyte sensor has been stored outside the specification range. In other words, in order to insert the analyte sensor into the body tissue, the insertion device has to engage with the analyte sensor, such as by a mechanical gripping of the analyte sensor by the insertion device, a loading of the analyte sensor into the insertion device, such as into a penetration element of the insertion device, or the like. The change in the at least one mechanical property of the analyte sensor induced by the fact that the storage conditions of the analyte sensor are outside a specification range may prevent this engagement, such as by changing the dimensions of the analyte sensor, by bending the analyte sensor or the like. Thus, as an example, the disabling device may be configured for changing a shape of the analyte sensor, the change of shape preventing the engagement of the analyte sensor by the insertion device.

Additionally or alternatively, the transcutaneous insertion itself may be prevented by the disabling device when the analyte sensor has been stored outside the specification range. Thus, the disabling device generally may also be configured for preventing the transcutaneous insertion of the at least one part of the analyte sensor when the analyte sensor has been stored outside the specification range. Various examples of preventing the transcutaneous insertion may be given and may be implemented in isolation or in combination.

The insertion device specifically may comprise at least one penetration element for penetrating into the body tissue of the user, specifically for penetrating the skin of the user. As used herein, the term “penetration element” as used herein is a broad term and is to be given its ordinary and customary meaning to a person of ordinary skill in the art and is not to be limited to a special or customized meaning. The term specifically may refer to an arbitrary element which is configured for penetrating the skin of the user and optionally for transporting the analyte sensor into the body tissue, through the skin of the user, during this penetration. For this purpose, the penetration element specifically may comprise at least one pointed or sharp part, such as a point and/or a blade. Specifically, the penetration element may be or may comprise at least one needle and/or at least one cannula.

The insertion device, as outlined above, may further comprise at least one actuator for driving the penetration element into the body tissue of the user. The actuator may directly or indirectly engage with the penetration element. The actuator may be actuated by manual actuation, such as by exerting a manual force onto at least one part of the actuator, such as onto at least one plunger of the actuator, the plunger being directly or indirectly connected with the penetration element, such that the movement of the plunger is transferred into the insertion movement of the penetration element. Additionally or alternatively, the actuator may comprise at least one transformation element, such as for transforming at least one type of stored energy into kinetic energy for driving the penetration element into the body tissue. As an example, the stored energy may be potential energy stored in a spring, wherein, when released, the spring exerts a force onto a plunger, the plunger driving the penetration element. Various other embodiments of the actuator are feasible and generally known to the skilled person in the field of in-vivo analyte measurements.

The actuator may comprise a plurality of parts, such as movable parts. As an example, the actuator may comprise at least one first part configured for being placed on the skin of the user and at least one second part which is movable against the first part, in order to drive the penetration element into the body tissue. The first part, as an example, may comprise at least one guide sleeve, e.g. a guide sleeve having a rim and/or another portion for direct or indirect placement onto the skin of the user. The second part, as an example, may comprise at least one plunger and/or at least one insertion sleeve movable with respect to the guide sleeve, e.g., an insertion sleeve placed around the guide sleeve and/or placed within the guide sleeve. As used herein, the term “guide sleeve” is a broad term and is to be given its ordinary and customary meaning to a person of ordinary skill in the art and is not to be limited to a special or customized meaning. The term specifically may refer to an element, specifically an element being part of an actuator, which is configured for guiding one or more other elements, such as by providing one or more guiding surfaces or guiding elements. The guide sleeve specifically may be or may comprise a hollow sleeve element, such as a tubular sleeve element. Similarly, the term “insertion sleeve” as used herein is a broad term and is to be given its ordinary and customary meaning to a person of ordinary skill in the art and is not to be limited to a special or customized meaning. The term specifically may refer to an element, specifically an element being part of the actuator, configured for driving an insertion as defined above. The insertion element specifically may be or may comprise at least one hollow sleeve element, such as a tubular sleeve element, which may be guided by the guide sleeve and which may directly or indirectly engage with the analyte sensor and/or the penetration element. Exemplary embodiments will be given below.

Thus, generally, the actuator may comprise at least one insertion sleeve for moving from a distal position to a proximal position thereby driving the penetration element into the body tissue of the user. The actuator may further comprise at least one guide sleeve for guiding the movement of the insertion sleeve.

In this set up, with the actuator comprising a guide sleeve and an insertion sleeve, the guide and insertion sleeves being movable with respect to each other, in order to drive the insertion movement, the disabling device specifically may be configured for preventing the relative movement of the sleeves. Thus, generally, the disabling device may be configured for preventing movement of the insertion sleeve from the distal position to the proximal position when the analyte sensor has been stored outside the specification range. For example, the disabling device may be arranged adjacent to at least one of the guide sleeve and the insertion sleeve, wherein, after undergoing the change in at least one mechanical property, the disabling device may be configured for blocking movement, specifically relative movement, of the insertion sleeve with respect to the guide sleeve. For example, the actuator may further comprise at least one cap connected with the insertion sleeve, specifically at least one cap at least partially enclosing one or more of the insertion sleeve, the guide sleeve and further components of the actuator. The cap may specifically be connected to the insertion sleeve such that, when the user applies a force to the cap, the cap together with the insertion sleeve is configured for moving from the distal position to the proximal position. The disabling device may be arranged adjacent to at least one of the cap and the guide sleeve, wherein, after undergoing the change in at least one mechanical property, the disabling device may be configured for blocking movement, specifically relative movement, of the cap with respect to the guide sleeve.

The actuator may comprise a plurality of further elements. Thus, as an example, the actuator may comprise one or more spring elements for various purposes. As an example, the actuator may comprise at least one spring element as an energy storage device. The spring element as an energy storage device may be configured for driving the insertion of the analyte sensor. In this set up, the disabling device may be configured for preventing actuation of the spring element by the user upon undergoing the change in the mechanical property by the monitoring element. For example, a release button of the spring element may be blocked or, alternatively or additionally, an actuation of the spring element prior to insertion may be caused. Additionally or alternatively, the actuator may comprise at least one spring element for retracting the penetration element from the body tissue after insertion. Thus, generally, the actuator may comprise at least one penetration element retractor for retracting the penetration element from the body tissue of the user after insertion of the analyte sensor into the body tissue of the user. The penetration element retractor may be configured for engaging with the penetration element when being moved from a proximal position to a distal position. The actuator may further comprise at least one elastic element, specifically at least one elastic spring element, arranged in between the penetration element retractor and the insertion sleeve, wherein the elastic element may be configured for exerting a force to the penetration element retractor after insertion of the analyte sensor into the body tissue of the user, thereby moving the penetration element retractor and the penetration element from the proximal position to the distal position. The disabling device specifically may be configured for releasing the elastic element prior to insertion of the analyte sensor into the body tissue of the user when the analyte sensor has been stored outside the specification range, thereby moving the penetration element retractor and the penetration element from the proximal position to the distal position.

As outlined above, the monitoring element is configured for undergoing a change in at least one mechanical property when storage conditions of the analyte sensor are outside a specification range. For this purpose, the monitoring element may comprise at least one material which is sensitive to the storage conditions. For example, the material may be or may comprise polyvinyl alcohol (PVA). As outlined in H. Hu et al., “Combined Effect of Relative Humidity and Temperature on Dynamic Viscoelastic Properties and Glass Transition of Poly(vinyl alcohol)” in J. Appl. Polym. Sci., 130:3161-3167, FIGS. 2 and 3, the mechanical properties of PVA may depend on temperature and/or humidity. Thus, a material comprising PVA may be sensitive to both storage conditions comprising temperature and/or humidity. As an example, various plastic materials may be used which are designed with respect to their sensitivity against temperature and/or humidity. For tailoring plastic materials with respect to their temperature sensitivity and/or humidity sensitivity, reference may be made e.g. to H. Hu et al., “Combined Effect of Relative Humidity and Temperature on Dynamic Viscoelastic Properties and Glass Transition of Poly(vinyl alcohol)” in J. Appl. Polym. Sci., 130:3161-3167, and/or to N. Jain et al., “A review on mechanical and water absorption properties of polyvinyl alcohol based composites/films” in Journal of the Mechanical Behavior of Materials, Vol. 26, no. 5-6, 2017, pp. 213-222. Specifically, the at least one plastic material may comprise polyvinyl alcohol (PVA). Thus, generally, the monitoring element may comprise or may even consist of PVA and/or a PVA blend. For example, the monitoring element may be or may comprise at least one PVA-based composite. The mechanical property of the PVA-based composite may be altered using fiber and/or particle reinforcement, as exemplarily summarized in N. Jain et al., “A review on mechanical and water absorption properties of polyvinyl alcohol based composites/films” in Journal of the Mechanical Behavior of Materials, Vol. 26, no. 5-6, 2017, pp. 213-222. For example, the monitoring element may comprise or may even consist of an element such as a pin and/or a rod which is fully or partially made of PVA, specifically a pin and/or a rod which is counterbalanced by the above-mentioned at least one counterbalancing element. PVA is rather sensitive against humidity and/or temperature, so the tensile strength of the pin and/or rod may change significantly in case the storage conditions are outside a specification range which is, as an example, prespecified and/or which is predetermined by the analyte sensor and/or the PVA material.

The disabling device may further comprise at least one indicator element configured for indicating that the transcutaneous insertion of the analyte sensor into the body tissue is irreversibly prevented. The indicator element may generally be or may comprise an arbitrary element or a combination of elements having at least two different states which are discernible by a user, e.g., by visual inspection, acoustically or optically, at least one of the states indicating that the storage conditions of the analyte sensor are within specification range and at least one of the states indicating that the storage conditions of the analyte sensor are outside the specification range. The indicator element generally may comprise at least one element selected from the group consisting of: an optical indicator element, specifically an optical indicator element configured for undergoing at least one color change when the analyte sensor has been stored outside the specification range; a mechanical indicator element. For example, the indicator element may at least partially be comprised by the disabling device such that when the transcutaneous insertion of the analyte sensor into the body tissue is irreversibly prevented, at least a part of the disabling device, specifically at least a part of the monitoring element, may be visible to the user. Additionally, the indicator element may be contrast colored, specifically to enhance visibility by the user. Alternatively or additionally, indicator elements separate from the disabling device may also be feasible.

The indicator element may fully or partially be coupled and/or integrated to the monitoring element. Thus, the monitoring element itself may fully or partially function as the indicator element, such as by making the change in the at least one mechanical property of the monitoring element detectable by the user, e.g., visually, acoustically or optically. Alternatively, the indicator element may also be separate from the monitoring element.

The medical system, as outlined above, may comprise a plurality of components. The medical system specifically may comprise at least one reusable part and at least one disposable part which may engage with each other, specifically reversibly. The reusable part, as an example, may comprise the insertion device. The at least one disabling device, specifically, may fully or partially be part of the disposable part of the medical system. The analyte sensor may be part of the disposable part or also may be part of the reusable part. Specifically, the disabling device may be configured for disabling an assembly of the reusable part and the disposable part when the analyte sensor has been stored outside the specification range. As an example, the disabling device may undergo a change in shape in case the analyte sensor's storage conditions are outside the specification range, so a coupling between the disposable part and the reusable part is prevented. The medical system, however, may be designed such that the transcutaneous insertion of the analyte sensor into the body tissue requires the coupling of the disposable part and the reusable part, so the preventing of the coupling of the disposable parts to the reusable part or vice versa may prevent the insertion of the analyte sensor.

In a further aspect of the present disclosure, a method for monitoring the storage conditions of at least one medical system is disclosed. The medical system comprises at least one analyte sensor for detecting at least one analyte in a bodily fluid. The analyte sensor is configured for at least partial transcutaneous insertion into a body tissue of a user. The method specifically may use a medical system in accordance with the present disclosure, such as according to any one of the embodiments described above and/or according to any one of the embodiments described in further detail below. Thus, generally, for possible definitions and/or options of the medical system as used in the method, reference may be made to the description of the medical system above or below.

The method comprises several method steps which may be performed in the order given below. It shall be noted, however, that another order is generally also possible. Further, it is also possible to perform two or more of the method steps simultaneously or in a fashion overlapping in time. Further, it is also feasible to perform one or more of the method steps permanently or repeatedly.

Thus, the method comprises operably connecting at least one disabling device to the analyte sensor. The disabling device comprises at least one monitoring element configured for undergoing a change in at least one mechanical property when storage conditions of the analyte sensor are outside a specification range. The mechanical property change is configured such that the transcutaneous insertion of the analyte sensor into the body tissue is irreversibly prevented. Thus, the method may further comprise irreversibly preventing the transcutaneous insertion of the analyte sensor into the body tissue, by using the disabling device, when the storage conditions of the analyte sensor are outside a specification range.

The medical system and the method for monitoring the storage conditions of at least one medical system according to the present disclosure may provide a large number of advantages compared to known methods and devices. Specifically, the medical system and the method according to the present disclosure may provide a mechanical prevention of insertion of the analyte sensor, e.g. by mechanically disabling the insertion device to insert the analyte sensor into the body tissue of the user. The disabling device may specifically comprise at least one mechanical part being made of the material which is sensitive to the storage conditions, such as temperature and/or humidity, thereby providing a simple disabling mechanism in the medical system, specifically in the insertion device. For example, a tensile strength of the temperature and/or humidity sensing material may drop when being exposed to temperatures and/or humidity outside the specification range, thus adsorbing a quantity of water exceeding the prespecified range. The intact disabling device may hold a spring compressed. When the medical system is stored in storage conditions outside the specification range, the disabling device may undergo a change in the mechanical strength due to temperature and/or humidity exposure, and, thus, may break and may release the spring. The spring may move at least one pin in the insertion device such that the insertion device is locked and insertion is prevented. Additionally, the medical system may comprise the at least one mechanical indicator element, specifically being visible from outside the medical system, and configured for indicating readiness or disability of the medical system.

An example of the material which is sensitive to the storage conditions may be polyvinyl alcohol. Specifically, as outlined above, PVA may change its tensile strength depending on both temperature and humidity. Generally, PVA may lose its modulus rapidly above a temperature of 50° C. and above relative humidity of 20-60% depending on temperature. The properties of the material may be fine-tuned by using PVA-based composites, such as by mixing with other polymers thereby changing a sensitivity to temperature and/or humidity. Reference may be made to above-identified scientific publications.

Further, the medical system and the method according to the present disclosure may provide a mechanical realization of preventing inserting the analyte sensor when the analyte sensor was stored in storage conditions outside the specification range. The disabling device and the analyte sensor may be stored in a common enclosure. Generally, in order to control humidity level, the medical system may be provided, e.g., for shipping, in a hermetically sealed bag, optionally with some humidity absorber. In case bag is damaged during shipping and/or storage, the medical system may be exposed to excess humidity and the disabling device, specifically comprising the material being sensitive to humidity, may start to adsorb the water irreversibly and may gradually undergo the changes in the mechanical property until it brakes and/or irreversibly disables insertion of the analyte sensor. The mechanical properties of the disabling device may specifically be tuned for a predefined integral humidity, specifically a humidity which critically influences performance of the analyte sensor. The temperature sensitivity may be considered in addition or as an alternative. In this example, a rise in temperature, e.g., once a predefined critical temperature is reached, the disabling device may undergo the change in the mechanical property and, thus, may prevent insertion of the analyte sensor. Temperature and humidity effects of the material of the disabling device may be combined to provide faster change in the mechanical property, which is of specific advantage for medical system comprising glucose monitoring biosensors which, generally, have faster fouling during storage in storage conditions outside the specification range.

As outlined above, the medical system may comprise the insertion device for at least partially inserting the analyte sensor, specifically a medical sensor, such as a glucose sensor, into a subject's skin. The medical system and the method according to the present disclosure may specifically provide prevention of the analyte sensor's insertion when the analyte sensor has been stored outside the specification range, specifically exposed to potentially harmful environmental conditions, such as too high temperatures and/or too high humidity. The medical system and the method according to the present disclosure comprises the disabling device and, thus, may integrate a simple disabling mechanism including a material that is sensitive to the environmental conditions, such as a temperature and/or humidity sensing material. For example, the medical system may comprise an integrated sensor-insertion-system where the analyte sensor may be supplied, sold and/or stored within the insertion device, wherein the disabling device may be part of the insertion device, specifically in such a way that the disabling device comprising the material being sensitive to temperature and/or humidity may yield a mechanically locking and/or destroying of the insertion device when exposed outside the specification range, specifically to certain potentially harmful environmental conditions. Alternatively, the analyte sensor may be supplied, sold and/or stored separate from the insertion device and/or at least separate from a part of the insertion device comprising the insertion mechanism. The disabling device may be part of or may be connected with the analyte sensor and may, as an example, mechanically prohibit the assembling of analyte sensor and the insertion device.

The medical system and the method according to the present disclosure may increase the safety of using analyte sensors since an improperly stored analyte sensor may lose sensitivity and, thus, may detect incorrect analyte concentration values. Additionally, by disabling the insertion device, unnecessary insertion of the faulty analyte sensor can be prevented.

Summarizing and without excluding further possible embodiments, the following embodiments may be envisaged:
Embodiment 1: A medical system, comprising:
at least one analyte sensor for detecting at least one analyte in a bodily fluid, the analyte sensor being configured for at least partial transcutaneous insertion into a body tissue of a user; and
at least one disabling device operably connected to the analyte sensor, the disabling device comprising at least one monitoring element, the monitoring element being configured for undergoing a change in at least one mechanical property when storage conditions of the analyte sensor are outside a specification range, the mechanical property change being configured such that the transcutaneous insertion of the analyte sensor into the body tissue is irreversibly prevented.
Embodiment 2: The medical system according to the preceding embodiment, wherein the mechanical change is selected from the group consisting of: a change in a tensile strength of the monitoring element; a change in a shape of the monitoring element; an elongation of the monitoring element under load, specifically an elongation of the monitoring element under spring load; a breaking of the monitoring element.
Embodiment 3: The medical system according to any one of the preceding embodiments, wherein the specification range of the storage conditions is defined by at least one of a predetermined temperature range and a predetermined humidity range.
Embodiment 4: The medical system according to any one of the preceding embodiments, wherein the disabling device further comprises at least one counterbalancing element, the counterbalancing element and the monitoring element exerting opposing forces onto at least one part of the disabling device.
Embodiment 5: The medical system according to the preceding embodiment, wherein the counterbalancing element comprises at least one spring element.
Embodiment 6: The medical system according to any one of the two preceding embodiments, wherein the opposing forces exerted by the counterbalancing element and the monitoring element are balanced when the analyte sensor has been stored under storage conditions within the specification range.
Embodiment 7: The medical system according to any one of the three preceding embodiments, wherein the opposing forces exerted by the counterbalancing element and the monitoring element are unbalanced when the analyte sensor has been stored, at least over a period of time, under storage conditions outside the specification range.
Embodiment 8: The medical system according to any one of the four preceding embodiments, wherein the at least one part of the disabling device onto which the opposing forces of the counterbalancing element and the monitoring element are exerted comprises at least one mechanical lock configured for moving from an unlocked position into a locked position when the opposing forces of the counterbalancing element and the monitoring element become unbalanced due to a storage of the analyte sensor outside the specification range.
Embodiment 9: The medical system according to any one of the preceding embodiments, further comprising: at least one insertion device configured for engaging with the analyte sensor, and for transcutaneously inserting at least a part of the analyte sensor into the body tissue.
Embodiment 10: The medical system according to the preceding embodiment, wherein the disabling device is configured for preventing an engagement of the analyte sensor by the insertion device when the analyte sensor has been stored outside the specification range.
Embodiment 11: The medical system according to the preceding embodiment, wherein the disabling device is configured for changing a shape of the analyte sensor, the change of shape preventing the engagement of the analyte sensor by the insertion device.
Embodiment 12: The medical system according to any one of the three preceding embodiments, wherein the disabling device is configured for preventing the transcutaneous insertion of the at least one part of the analyte sensor when the analyte sensor has been stored outside the specification range.
Embodiment 13: The medical system according to any one of the four preceding embodiments, wherein the insertion device comprises at least one penetration element, specifically at least one cannula, for penetrating into the body tissue of the user, specifically for penetrating a skin of the user, and wherein the insertion device further comprises at least one actuator for driving the penetration element into the body tissue of the user.
Embodiment 14: The medical system according to the preceding embodiment, wherein the actuator comprises at least one insertion sleeve for moving from a distal position to a proximal position thereby driving the penetration element into the body tissue of the user, and wherein the actuator further comprises at least one guide sleeve for guiding movement of the insertion sleeve.
Embodiment 15: The medical system according to the preceding embodiment, wherein the disabling device is configured for preventing movement of the insertion sleeve from the distal position to the proximal position when the analyte sensor has been stored outside the specification range.
Embodiment 16: The medical system according to the preceding claim, wherein the disabling device is arranged adjacent to at least one of the guide sleeve and the insertion sleeve, wherein, after undergoing the change in at least one mechanical property, the disabling device is configured for blocking movement of the insertion sleeve with respect to the guide sleeve.
Embodiment 17: The medical system according to any one of the two preceding claims, wherein the actuator comprises at least one cap connected with the insertion sleeve, wherein the disabling device is arranged adjacent to at least one of the cap and the guide sleeve, wherein, after undergoing the change in at least one mechanical property, the disabling device is configured for blocking movement of the cap with respect to the guide sleeve.
Embodiment 18: The medical system according to any one of the five preceding embodiments, wherein the actuator comprises at least one penetration element retractor for retracting the penetration element from the body tissue of the user after insertion of the analyte sensor into the body tissue of the user, wherein the penetration element retractor is configured for engaging with the penetration element when being moved from a proximal position to a distal position.
Embodiment 19: The medical system according to the preceding embodiment, wherein the actuator further comprises at least one elastic element, specifically at least one elastic spring element, arranged in between the penetration element retractor and the insertion sleeve, wherein the elastic element is configured for exerting a force to the penetration element retractor after insertion of the analyte sensor into the body tissue of the user thereby moving the penetration element retractor and the penetration element from the proximal position to the distal position.
Embodiment 20: The medical system according to the preceding embodiment, wherein the disabling device is configured for releasing the elastic element prior to insertion of the analyte sensor into the body tissue of the user when the analyte sensor has been stored outside the specification range thereby moving the penetration element retractor and the penetration element from the proximal position to the distal position.
Embodiment 21: The medical system according to any one of the preceding embodiments, wherein the monitoring element comprises, specifically consists of, polyvinyl alcohol (PVA) and/or a PVA blend.
Embodiment 22: The medical system according to any one of the preceding embodiments, wherein the disabling device further comprises at least one indicator element configured for indicating that the transcutaneous insertion of the analyte sensor into the body tissue is irreversibly prevented.
Embodiment 23: The medical system according to the preceding embodiment, wherein the indicator element comprises at least one element selected from the group consisting of: an optical indicator element, specifically an optical indicator element configured for undergoing at least one color change when the analyte sensor has been stored outside the specification range; a mechanical indicator element.
Embodiment 24: The medical system according to any one of the preceding embodiments, wherein the medical system comprises at least one reusable part and at least one disposable part, the disposable part comprising at least the disabling device.
Embodiment 25: The medical system according to the preceding embodiment, wherein the disabling device is configured for disabling an assembly of the reusable part and the disposable part when the analyte sensor has been stored outside the specification range.
Embodiment 26: A method for monitoring the storage conditions of at least one medical system comprising at least one analyte sensor for detecting at least one analyte in a bodily fluid, the analyte sensor being configured for at least partial transcutaneous insertion into a body tissue of a user, the method comprising operably connecting at least one disabling device to the analyte sensor, the disabling device comprising at least one monitoring element, the monitoring element being configured for undergoing a change in at least one mechanical property when storage conditions of the analyte sensor are outside a specification range, the mechanical property change being configured such that the transcutaneous insertion of the analyte sensor into the body tissue is irreversibly prevented.
Embodiment 27: The method according to the preceding embodiment, wherein a medical system according to any one of the preceding embodiments referring to a medical system is used.

### BRIEF DESCRIPTION OF THE DRAWINGS

Further optional features and embodiments will be disclosed in more detail in the subsequent description of embodiments. Therein, the respective optional features may be realized in an isolated fashion as well as in any arbitrary feasible combination, as the skilled person will realize. The scope of the invention is not restricted by the preferred embodiments. The embodiments are schematically depicted in the Figures. Therein, identical reference numbers in these Figures refer to identical or functionally comparable elements.

In the Figures:

FIG. 1 shows an embodiment of a medical system in a sectional view;

FIGS. 2A and 2B show a detailed sectional view of an embodiment of the disabling device of the medical system; and

FIG. 3 shows a flow chart of an embodiment of a method for monitoring the storage conditions of at least one medical system.

### DETAILED DESCRIPTION

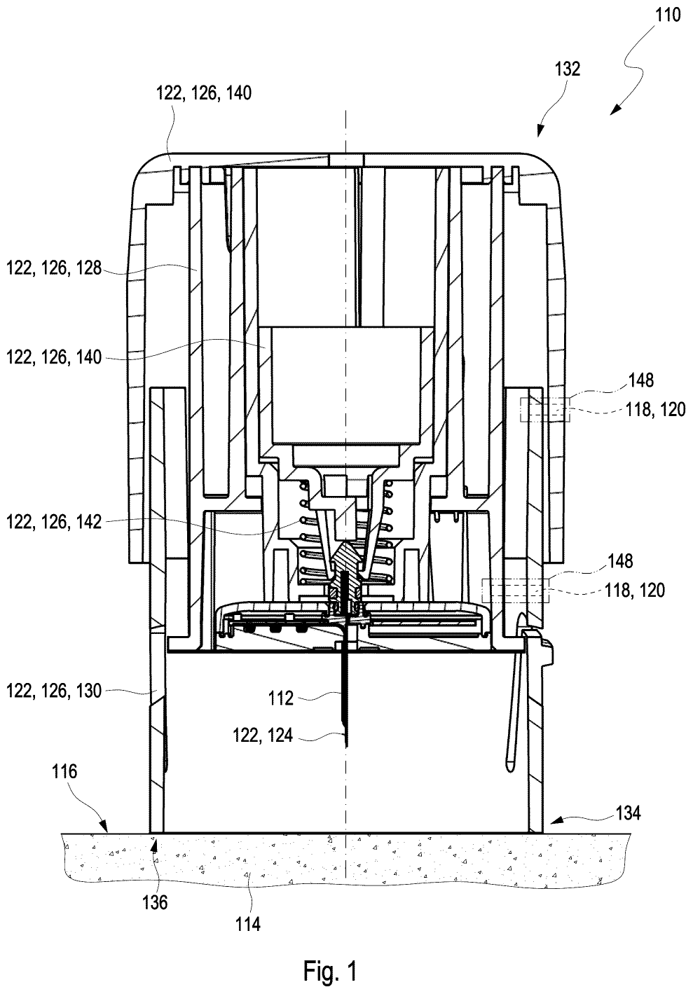

FIG. 1 shows an exemplary embodiment of a medical system 110 in a sectional view. In FIG. 1, the medical system 110 is shown in a longitudinal-sectional view, wherein the longitudinal-sectional view passes through a median plane of the medical system 110. Further, FIG. 1 shows the medical system 110 prior to insertion of at least one analyte sensor 112.

The medical system 110 comprises the at least one analyte sensor 112 for detecting at least one analyte in a bodily fluid, the analyte sensor 112 being configured for at least partial transcutaneous insertion into a body tissue 114 of a user 116. The analyte sensor 112 specifically may be configured for being used in qualitatively and/or quantitatively detecting the at least one analyte in the bodily fluid. For example, the analyte to be detected may be glucose. The analyte sensor 112 specifically may be configured for continuously detecting the analyte in the bodily fluid present in the body tissue 114 of the user 116. Specifically, the analyte sensor 112 may be an in-vivo analyte sensor. Thus, in this example, the medical system 110 may specifically be a continuous glucose monitoring (CGM) system. However, other analytes to be detected and/or other use cases for the medical system 110 are also feasible.

The medical system 110 further comprises at least one disabling device 118 operably connected to the analyte sensor 112. The disabling device 118 comprises at least one monitoring element 120, the monitoring element 120 being configured for undergoing a change in at least one mechanical property when storage conditions of the analyte sensor 112 are outside a specification range, the mechanical property change being configured such that the transcutaneous insertion of the analyte sensor 112 into the body tissue 114 is irreversibly prevented.

As can be seen in FIG. 1, the medical system 110 may further comprise at least one insertion device 122 configured for engaging with the analyte sensor 112, and for transcutaneously inserting at least a part of the analyte sensor 112 into the body tissue 114. The insertion device 122 specifically may comprise at least one penetration element 124 for penetrating into the body tissue 114 of the user 116, specifically for penetrating a skin of the user 116. Specifically, the penetration element 124 may be or may comprise at least one needle and/or at least one cannula.

The insertion device 122 may further comprise at least one actuator 126 for driving the penetration element 124 into the body tissue 114 of the user 116. In the exemplary embodiment of FIG. 1, the actuator 126 may comprise at least one insertion sleeve 128 and at least one guide sleeve 130. The insertion sleeve 128 may be configured for moving from a distal position 132 to a proximal position 134 thereby driving the penetration element 124 into the body tissue 114 of the user 116. The guide sleeve 130 may be configured for guiding the movement of the insertion sleeve 128. For example, the insertion sleeve 128 and the guide sleeve 130 may be movable parts of the actuator 126, specifically being movable with respect to each other. As can be seen in FIG. 1, the insertion sleeve 128 may be placed within the guide sleeve 130. The guide sleeve 130 may have a rim 136 and may be configured for being placed directly on the skin of the user 116. The insertion sleeve 128 may be movable against the guide sleeve 130 in order to drive the penetration element 124 into the body tissue 114.

In this set up, with the actuator 126 comprising the guide sleeve 130 and the insertion sleeve 128, the guide 130 and insertion sleeves 128 being movable with respect to each other, in order to drive the insertion movement, the disabling device 118 specifically may be configured for preventing the relative movement of the sleeves. Thus, generally, the disabling device 118 may be configured for preventing movement of the insertion sleeve 128 from the distal position 132 to the proximal position 134 when the analyte sensor 112 has been stored outside the specification range. For example, as shown in FIG. 1, the disabling device 118 may be arranged adjacent to at least one of the guide sleeve 130 and the insertion sleeve 128, wherein, after undergoing the change in at least one mechanical property, the disabling device 118 may be configured for blocking movement, specifically relative movement, of the insertion sleeve 128 with respect to the guide sleeve 130. Alternatively or additionally, as exemplarily shown in FIG. 1, the actuator 126 may further comprise at least one cap 138 connected with the insertion sleeve 128, specifically at least one cap 138 at least partially enclosing the insertion sleeve 128, the guide sleeve 130 and/or further components of the actuator 126, as will be outlined in further detail below. The cap 138 may specifically be connected to the insertion sleeve 128 such that, when the user 116 applies a force to the cap 138, the cap 138 together with the insertion sleeve 128 is configured for moving from the distal position 132 to the proximal position 134. The disabling device 118 may be arranged adjacent to at least one of the cap 138 and the guide sleeve 130, wherein, after undergoing the change in at least one mechanical property, the disabling device 118 may be configured for blocking movement, specifically relative movement, of the cap 138 with respect to the guide sleeve 130.

Further, in the exemplary embodiment of FIG. 1, the actuator 126 may comprise at least one penetration element retractor 140 for retracting the penetration element 124 from the body tissue 114 of the user 116 after insertion of the analyte sensor 112 into the body tissue 114 of the user 116. The penetration element retractor 140 may be configured for engaging with the penetration element 124 when being moved from the proximal position 134 to the distal position 132. The actuator 126 may further comprise at least one elastic element 142, specifically at least one elastic spring element, arranged in between the penetration element retractor 140 and the insertion sleeve 128, wherein the elastic element 142 may be configured for exerting a force to the penetration element retractor 140 after insertion of the analyte sensor 112 into the body tissue 114 of the user 116, thereby moving the penetration element retractor 140 and the penetration element 124 from the proximal position 134 to the distal position 132. Additionally or alternatively, the disabling device 118 specifically may be configured for releasing the elastic element 142 prior to insertion of the analyte sensor 112 into the body tissue 114 of the user 116 when the analyte sensor 112 has been stored outside the specification range, thereby moving the penetration element retractor 140 and the penetration element 124 from the proximal position 134 to the distal position 132. In this example, as shown in FIG. 1, the penetration element retractor 140 may be positioned within the insertion sleeve 128 prior to insertion of the analyte sensor 112, specifically in a locking state such that the elastic element 142 may not be able to move the penetration element retractor 140 and the penetration element 124 from the proximal position 134 to the distal position 132. Upon movement of insertion sleeve 128 from the distal position 132 to the proximal position 134, specifically yielding the insertion of the analyte sensor 112 into the body tissue 114 of the user 116, the penetration element retractor 140 may be released and the elastic element 142 may be configured for exerting the force to the penetration element retractor 140 thereby moving the penetration element retractor 140 and the penetration element 124 from the proximal position 134 to the distal position 132. The disabling device 118 may specifically be configured for releasing the penetration element retractor 140 prior to insertion of the analyte sensor 112 into the body tissue 114 of the user 116 when the analyte sensor 112 has been stored outside the specification range, which, in the sectional view of FIG. 1, is not visible.

The examples of the disabling device 118 described with respect to FIG. 1 may only represent exemplary embodiments of the disabling device 118 of the medical system 110. Further, the medical system 110 may comprise one, two or even all of these examples of the disabling device 118. Thus, generally, the examples of the disabling device 118 can be implemented in isolation or in combination in the medical system 110 according to the present disclosure.

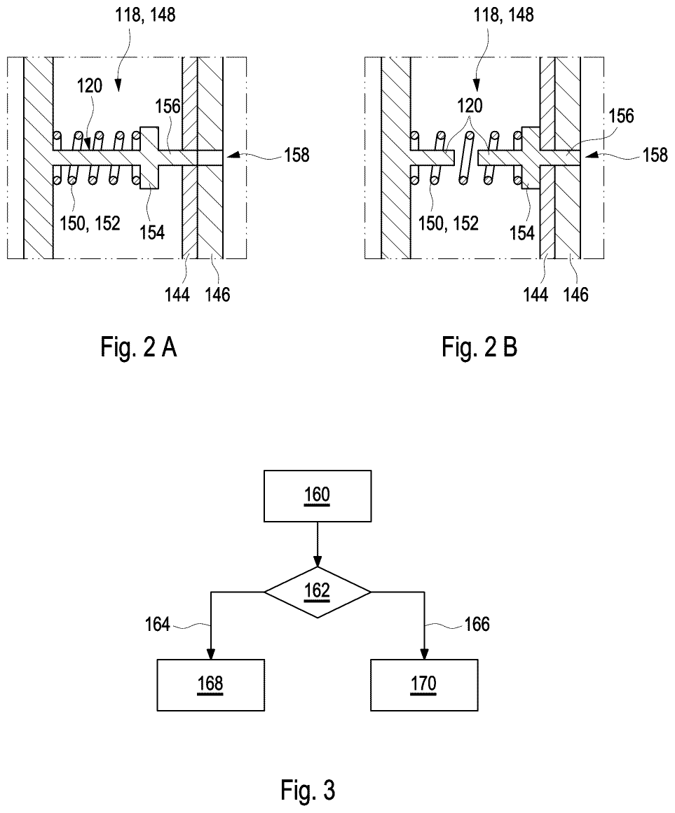

FIGS. 2A and 2B show a detailed sectional view of an exemplary embodiment of the disabling device 118 of the medical system 110. The exemplary embodiment of the disabling device 118 may be arranged adjacent to at least one inner wall 144 and at least one outer wall 146, wherein the inner wall 144 may be part of the insertion sleeve 128 or the guide sleeve 130, wherein the outer wall 146 may be part of the guide sleeve 130 or the cap 138, respectively, as indicated by reference number 148 in FIG. 1 indicating the detailed views of FIGS. 2A and 2B in the medical system 110. Further, FIG. 2A shows the disabling device 118 prior to the change in the at least one mechanical property, wherein FIG. 2B shows the disabling device 118 after the change in the at least one mechanical property.

As can be seen in FIG. 2A, the disabling device 118 may further comprise at least one counterbalancing element 150, the counterbalancing element 150 and the monitoring element 120 exerting opposing forces onto at least one part of the disabling device 118. In the exemplary embodiment of FIGS. 2A and 2B, the counterbalancing element 150 may comprise at least one spring element 152, specifically at least one spring element 152 being directly compressed by the monitoring element 120. Further, the at least one part of the disabling device 118 onto which the opposing forces of the counterbalancing element 150 and the monitoring element 120 are exerted may comprise at least one mechanical lock 154 configured for moving from an unlocked position (FIG. 2A) into a locked position (FIG. 2B) when the opposing forces of the counterbalancing element 150 and the monitoring element 120 become unbalanced due to a storage of the analyte sensor 112 outside the specification range. In this example, the mechanical lock 154 may specifically comprise at least one pin 156 configured for at least partially penetrating both the inner wall 144 and the outer wall 146 when moving from the unlocked position into the locked position.

In the situation of FIG. 2A, the opposing forces exerted by the counterbalancing element 150 and the monitoring element 120 may be balanced when the analyte sensor 112 has been stored under storage conditions within the specification range. Thus, the balancing of the opposing forces may be an indicator of the fact that the analyte sensor 112 has been stored under storage conditions within the specification range.

The monitoring element 120 may, as an example, comprise or even consist of PVA. As outlined above, PVA is rather sensitive against humidity and/or temperature, so the tensile strength of the monitoring element 120 may change significantly in case the storage conditions are outside the specification range, e.g. humidity and/or temperature of the storage conditions are outside the specification range.

Thus, in case temperature and/or humidity of the storage conditions of the analyte sensor 112 are outside the specification range, the tensile strength of the monitoring element 120 may decrease significantly. This situation is shown in FIG. 2B. Therein, the opposing forces exerted by the counterbalancing element 150 and the monitoring element 120 may become unbalanced when the analyte sensor 112 has been stored, at least over a period of time, under storage conditions outside the specification range. Thus, the change in the tensile strength of the monitoring element 120 due to the fact that storage conditions of the analyte sensor 112 are outside a specification range may lead to a breaking of the monitoring element 120. Alternatively or additionally, the change in the tensile strength of the monitoring element 120 due to the fact that storage conditions of the analyte sensor 112 are outside a specification range may lead to a change in shape of the monitoring element 120, e.g., a stretching of the monitoring element 120. As shown in FIG. 2B, by the net force of the counterbalancing element 150, the mechanical lock 154 may be moved in order to prevent the insertion of the analyte sensor 112. Specifically, the pin 156 may at least partially penetrate both the inner wall 144 and the outer wall 146 and, thus, a blocking effect may occur. The outer wall 146 may not be movable anymore with respect to the inner wall 144.

Further, as can be seen in FIGS. 2A and 2B, the disabling device 118 may further comprise at least one indicator element 158 configured for indicating that the transcutaneous insertion of the analyte sensor 112 into the body tissue 114 is irreversibly prevented. In this example, the indicator element 158 may be a mechanical indicator element. Specifically, the disabling device 118 comprising the mechanical lock 154 with the pin 156 may function as the indicator element 158 by making the change in the at least one mechanical property of the monitoring element 120 visually detectable by the user 116. For example, the pin 156, in the locked position, may be visible to the user 116. Other indicator elements, such as optical indicator element, may be feasible in addition or as an alternative.

FIG. 3 shows a flow chart of an exemplary embodiment of a method for monitoring the storage conditions of at least one medical system 110. The medical system 110 comprises the at least one analyte sensor 112 for detecting at least one analyte in a bodily fluid. The analyte sensor 112 is configured for at least partial transcutaneous insertion into the body tissue 114 of the user 116. The method specifically may use the medical system 110 according to the present disclosure, such as according to the exemplary embodiment of the medical system 110 shown in FIG. 1. Thus, for a detailed description of the medical system 110, reference may be made to the description of FIG. 1.

The method comprises several method steps which may be performed in the order given below. It shall be noted, however, that another order is generally also possible. Further, it is also possible to perform two or more of the method steps simultaneously or in a fashion overlapping in time. Further, it is also feasible to perform one or more of the method steps permanently or repeatedly.

The method comprises operably connecting the at least one disabling device 118 to the analyte sensor 112 (denoted by reference number 160). The disabling device 118 comprises the at least one monitoring element 120 configured for undergoing a change in the at least one mechanical property when storage conditions of the analyte sensor 112 are outside the specification range. The mechanical property change is configured such that the transcutaneous insertion of the analyte sensor 112 into the body tissue 114 is irreversibly prevented. During storage of the assembled medical system 110 (denoted by reference number 162), the storage conditions of the analyte sensor 112 are either outside the specification range (denoted by reference number 164) or remain in the specification range (denoted by reference number 166). Thus, the method may further comprise irreversibly preventing the transcutaneous insertion of the analyte sensor 112 into the body tissue 114, by using the disabling device 118, when the storage conditions of the analyte sensor 112 are outside a specification range (denoted by reference number 168). Alternatively, in case the storage conditions of the analyte sensor 112 remain in the specification range, the medical system 110 may remain in the as-manufactured state, specifically being configured, e.g. by using the insertion device 122, for transcutaneously inserting at least a part of the analyte sensor 112 into the body tissue 114 of the user 116 (denoted by reference number 170).

### List of reference numbers

110 medical system
112 analyte sensor
114 body tissue
116 user
118 disabling device
120 monitoring element
122 insertion device
124 penetration element
126 actuator
128 insertion sleeve
130 guide sleeve
132 distal position
134 proximal position
136 rim
138 cap
140 penetration element retractor
142 elastic element
144 inner wall
146 outer wall
148 detailed view of FIGS. 2A and 2B
150 counterbalancing element
152 spring element
154 mechanical lock
156 pin
158 indicator element
160 operably connecting the disabling device to the analyte sensor
162 storage
164 storage conditions outside the specification range
166 storage conditions in the specification range
168 irreversibly preventing the transcutaneous insertion of the analyte sensor
170 medial system remain in the as-manufactured state

While this invention has been described as having an exemplary design, the present invention may be further modified within the spirit and scope of this disclosure. This application is therefore intended to cover any variations, uses, or adaptations of the invention using its general principles.

What is claimed is:

1. A medical system, comprising:
at least one analyte sensor for detecting at least one analyte in a bodily fluid, the analyte sensor being configured for at least partial transcutaneous insertion into a body tissue of a user; and
at least one disabling device operably connected to the analyte sensor, the disabling device comprising at least one monitoring element, the monitoring element being configured for undergoing a change in at least one mechanical property when storage conditions of the analyte sensor are outside a specification range, the change in the mechanical property being configured such that the transcutaneous insertion of the analyte sensor into the body tissue is irreversibly prevented.

2. The medical system according to claim 1, wherein the change in the mechanical property is selected from the group consisting of: a change in a tensile strength of the monitoring element; a change in a shape of the monitoring element; an elongation of the monitoring element under load; a breaking of the monitoring element.

3. The medical system according to claim 1, wherein the specification range of the storage conditions is defined by at least one of a predetermined temperature range and a predetermined humidity range.

4. The medical system according to claim 1, wherein the disabling device further comprises at least one counterbalancing element, the counterbalancing element and the monitoring element exerting opposing forces onto at least one part of the disabling device, wherein the opposing forces exerted by the counterbalancing element and the monitoring element are unbalanced when the analyte sensor has been stored, at least over a period of time, under storage conditions outside the specification range.

5. The medical system according to claim 4, wherein the at least one part of the disabling device onto which the opposing forces of the counterbalancing element and the monitoring element are exerted comprises at least one mechanical lock configured for moving from an unlocked position into a locked position when the opposing forces of the counterbalancing element and the monitoring element become unbalanced due to a storage of the analyte sensor outside the specification range.

6. The medical system according to claim 1, further comprising:
at least one insertion device configured for engaging with the analyte sensor, and for transcutaneously inserting at least a part of the analyte sensor into the body tissue.

7. The medical system according to claim 6, wherein the disabling device is configured for preventing an engagement of the analyte sensor by the insertion device when the analyte sensor has been stored outside the specification range, wherein the disabling device is configured for changing a shape of the analyte sensor, the change of shape preventing the engagement of the analyte sensor by the insertion device.

8. The medical system according to claim 6, wherein the disabling device is configured for preventing the transcutaneous insertion of the at least one part of the analyte sensor when the analyte sensor has been stored outside the specification range.

9. The medical system according to claim 6, wherein the insertion device comprises at least one penetration element for penetrating into the body tissue of the user and at least one actuator for driving the penetration element into the body tissue of the user.

10. The medical system according to claim 9, wherein the actuator comprises at least one insertion sleeve movable from a distal position to a proximal position thereby driving the penetration element into the body tissue of the user, wherein the actuator further comprises at least one guide sleeve for guiding movement of the insertion sleeve, wherein the disabling device is configured for preventing movement of the insertion sleeve from the distal position to the proximal position when the analyte sensor has been stored outside the specification range.

11. The medical system according to claim 9, wherein the actuator comprises at least one insertion sleeve movable from a distal position to a proximal position thereby driving the penetration element into the body tissue of the user and at least one penetration element retractor for retracting the penetration element from the body tissue of the user after insertion of the analyte sensor into the body tissue of the user, wherein the penetration element retractor is configured for engaging with the penetration element when being moved from a proximal position to a distal position, wherein the actuator further comprises at least one elastic element, arranged in between the penetration element retractor and the insertion sleeve, wherein the elastic element is configured for exerting a force to the penetration element retractor after insertion of the analyte sensor into the body tissue of the user thereby moving the penetration element retractor and the penetration element from the proximal position to the distal position, wherein the disabling device is configured for releasing the elastic element prior to insertion of the analyte sensor into the body tissue of the user when the analyte sensor has been stored outside the specification range thereby moving the penetration element retractor and the penetration element from the proximal position to the distal position.

12. The medical system according to claim 1, wherein the monitoring element comprises a polyvinyl alcohol (PVA) and/or a PVA blend.

13. The medical system according to claim 1, wherein the disabling device further comprises at least one indicator element configured for indicating that the transcutaneous insertion of the analyte sensor into the body tissue is irreversibly prevented.

14. The medical system according to claim 1, wherein the medical system comprises at least one reusable part and at least one disposable part, the disposable part comprising at least the disabling device, wherein the disabling device is configured for disabling an assembly of the reusable part and the disposable part when the analyte sensor has been stored outside the specification range.

15. A method for monitoring the storage conditions of at least one medical system comprising:
providing at least one analyte sensor for detecting at least one analyte in a bodily fluid, the analyte sensor being configured for at least partial transcutaneous insertion into a body tissue of a user; and
operably connecting at least one disabling device to the analyte sensor, the disabling device comprising at least one monitoring element, the monitoring element being configured for undergoing a change in at least one mechanical property when storage conditions of the analyte sensor are outside a specification range, the change in the mechanical property being configured such that the transcutaneous insertion of the analyte sensor into the body tissue is irreversibly prevented.
